(19) United States
(12) Patent Application Publication
(10) Pub. No.: US 2011/0071090 A1
(43) Pub. Date: Mar. 24, 2011

(54) Method of mitigating adverse drug events using omega-3-fatty acids as a parenteral therapeutic drug vehicle

(75) Inventor: David F. Driscoll, Bridgewater, MA (US)

(73) Assignee: STABLE SOLUTIONS LLC, Goleta, CA (US)

(21) Appl. No.: 12/923,257

(22) Filed: Sep. 10, 2010

Related U.S. Application Data
(63) Continuation-in-part of application No. 12/382,196, filed on Mar. 11, 2009.
Application No. 12/382,196 is a continuation-in-part of application No. PCT/US2010/000723, filed on Mar. 11, 2010.
Application No. PCT/US2010/000723 is a continuation-in-part of application No. 12/382,196, filed on Mar. 11, 2009.

(51) Int. Cl. A61K 31/202; A61K 31/7048; A61K 31/343; A61K 31/52; A61K 38/13; A61K 31/436; A61K 31/405; A61K 31/407; A61P 29/00; A61P 43/00; A61P 37/04; A61P 35/00; A61P 7/10

(52) U.S. Cl. 514/20.5; 514/31; 514/469; 514/263.2; 514/291; 514/420; 514/413; 514/560

(57) ABSTRACT

A method of parenterally administering a composition, the method including parenterally administering to a person a composition including at least one omega-3 fatty acid and at least one drug, wherein the at least one omega-3 fatty acid source and the at least one drug are administered simultaneously.

### CROSS-REFERENCE TO RELATED APPLICATIONS

This application claims the benefit of priority and is a continuation-in-part of U.S. application Ser. No. 12/382,196 filed on Mar. 11, 2009, and is a continuation-in-part of International Application No. PCT/US2010/000723 filed on Mar. 11, 2010, which in turn is a continuation-in-part of U.S. application Ser. No. 12/382,196 filed on Mar. 11, 2009, the entire contents of both of which are incorporated by reference herein.

### BACKGROUND

1. Field

The present disclosure relates to medicinal formulations that can, for example, contain sufficient amounts of parenteral omega-3 fatty acids derived from naturally-occurring marine oils, and that can function as a novel “therapeutic” drug carrier, or vehicle. This proposed novel application is in contrast to conventional “pharmaceutical” drug carriers, or vehicles.

The marine oil-containing formulations can be in the form of an emulsion drug vehicle, comprising omega-3 fatty acids, attached to triglyceride or ester molecules, as an oil component of the emulsion, in addition to a water component. These two components of the emulsion, with the aid of a suitable surfactant, can exist as separate, but miscible phases, along with one or more drugs that, when parenterally administered without accompanying omega-3 fatty acids-containing marine oil, would often be expected to cause collateral damage to a vital organ. The novel marine oil-containing formulation can be given by intravenous administration, as an oil-in-water emulsion containing the drug(s). The addition of the omega-3 fatty acids (for example, eicosapentaenoic acid (EPA), docosahexaenoic acid (DHA) and/or docosapentaenoic acid (DPA)) to formulations containing selected drugs can reduce at least one adverse event profile of those drugs upon intravenous administration. The at least one adverse event profile can result from a drug toxicity, and can be manifested by oxidative stress, inflammation, immune stimulation or ischemia of one or more vital organs, or a combination thereof.

2. Related Art

Bioactive omega-3, or n3, fatty acids (n3-FAs) are present in naturally-occurring marine oil triglycerides and are contained in a variety of commercial products as nutritional supplements, in such forms, for example, as soft gelatin capsules, foods, enteral nutrition formulations, and parenteral oil-in-water nutrition emulsions. As well, semi-synthetically-derived n3-FAs also exist in a highly purified form, such as omega-3 acid ethyl esters in liquid-filled capsules, used for the treatment of hypertriglyceridemia. The bioactive components of marine oils can consist of three main omega-3 fatty acids: namely, eicosapentaenoic acid (EPA), docosahexaenoic acid (DHA) and, to a lesser extent, docosapentaenoic acid (DPA).

In the critical care setting, the administration of clinical nutrition supplemented with omega-3 fatty acids in fish oil-containing lipid injectable emulsions has been shown to reduce mortality, the use of antibiotics and the length of hospital stay. See Heller et al, 2006; Wichmann et al, 2007. These general beneficial effects were observed in acutely ill surgical patients, but the specific reasons for these positive findings were not clear, as noted by the following excerpt from one of the study conclusions: “In view of the lack of substantial study literature concerning diagnosis-related nutritional single-substrate intervention in the critically ill, the present data can be used in formulating hypotheses . . . ” (Heller et al., 2006). In other words, there is evidence to support the general, or nonspecific, clinical benefits of providing n3-FAs to acutely ill patients, but the reasons for these benefits are poorly understood.

By comparison, in critically ill medical patients, supplementation with fish oil parenteral nutrition emulsions did not affect inflammation or outcome. See Frieseckes et al, 2008. Finally, in a recent review about the role of fish oil-containing parenteral nutrition emulsions, the following statement summarizes their present status in clinical medicine: “ . . . the influence on inflammatory processes, immune function and clinical endpoints is not clear, since there are too few studies and those that are available report contradictory findings”. See Calder, 2010a. Due to the heterogeneity of patient populations, and the complex array of diseases and treatments, present application of n3-FAs as such, is non-specific. Moreover, there are significant qualitative differences with respect to available fish oil emulsions and various oil compositions (Driscoll, 2009, 2010), further masking any potential clinical benefits.

### SUMMARY

An exemplary embodiment can address a significant aspect in the treatment of acutely ill patients requiring intravenous support, namely drug therapy, which can be a significant contributor to determining clinical outcome. Intravenous therapies can be prescribed in various settings (for example, hospital, ambulatory care, hospice, nursing home, rehabilitation or home) depending upon the patient, the disease and the prognosis. The co-administration of a parenteral drug known to cause damage to vital organs, but now accompanied by specific n3-FAs as a therapeutic drug vehicle, at the onset of medication therapy, can allow rapid incorporation of n3-FAs into plasma cell membranes. The n3-FAs can replace n6-FAs present from typical dietary sources, and therefore the former can reduce injury to these vital organs, for example, by altering the production of lipid mediators produced, and, likely improving clinical outcomes.

According to an exemplary aspect, a method of parenterally administering a composition is provided, the method comprising parenterally administering to a person a composition comprising at least one omega-3 fatty acid and at least one drug, wherein the at least one omega-3 fatty acid and the at least one drug are administered simultaneously.

According to an exemplary aspect, a composition can contain bioavailable amounts of naturally- or synthetically-derived omega-3 fatty acids (i.e., n3-fatty acids, or n3-FAs). The omega-3 fatty acids can be present in a sufficient concentration as a pharmaceutical product in order to provide a therapeutic benefit, along with a prescribed drug whose side effect profile is associated with significant damage to vital organs. This combination can form a unique “therapeutic drug vehicle”, or “TDV”. See Driscoll, 2009, Driscoll, 2010.

According to another exemplary aspect, a composition containing both at least one prescribed drug and n3-FA-containing oil, as an oil-containing injectable emulsion, is administered by an intravenous route of administration.

According to another exemplary aspect, a combined intravenous administration of concentrated n3-FAs and at least one prescribed drug known to cause collateral damage to vital organs in a single injectable formulation can ensure the highest bioavailability and rapid incorporation of n3-FAs into plasma membranes not achievable by the oral or enteral routes of administration.

According to another exemplary aspect, at least one adverse consequence of drug toxicity to vital organs can be ameliorated or eliminated by the pharmacological actions of the n3-FAs, EPA, DHA and/or DPA, which can act to reduce organ injury from pronounced oxidative stress, inflammation, immune modulation, and/or ischemia affecting one or more vital organs.

According to another exemplary aspect, amounts of bioactive n3-FAs, i.e., the sum of EPA, DHA and/or DPA, ranging in total concentration from 1 to 300 mg/kg, are present in a parenteral formulation in order to mitigate damage to one or more vital organs caused by the at least one prescribed drug that is/are also present in the composition.

According to another exemplary aspect, the type(s) and relative amounts of bioactive n3-FAs present in the formulation can vary from 0 to 100% for each n3-FA oil component, for example, EPA, DHA and DPA, for a given total concentration of all n3-FA oil components.

According to another exemplary aspect, the effective combination (EPA±DHA±DPA) and dose of n3-FAs (1 mg/kg to 300 mg/kg), along with each prescribed drug in the injectable formulation can be n3FA+drug-specific, n3-FA+drug category-specific, or apply to a broad spectrum of drugs that respond to a specific combination-dose n3-FA regimen.

According to another exemplary aspect, the prescribed drug can reside in either the oil fraction or the water fraction of the injectable formulation, i.e., within the “dispersed” (i.e., “internal”) or “continuous” (i.e., “external”) phase of an emulsion, depending on whether the drug is oil-soluble or water-soluble, respectively. Accordingly, the lack of pharmaceutical consequence of the location of the drug, i.e., whether it resides in the dispersed or continuous phase, is novel in this disclosure. That is, the drug in most traditional drug-based injectable emulsions is usually water-insoluble, and therefore it necessarily almost always resides in the dispersed (oil) phase. Consequently, such emulsions primarily serve only as drug carriers, or pharmaceutical drug vehicles. In contrast, in the present disclosure, the n3-FA oil-containing “carrier” itself can play an active pharmacological role as a therapeutic drug vehicle, independent of whether the drug resides in the oil or water fraction of the emulsion, for example, regardless of whether it is located in the internal (dispersed) or external (continuous) phase of the emulsion.

According to another exemplary aspect, the n3-FAs combined with a particular drug can accentuate the pharmacological actions of the intended drug therapy, independent of the role of the n3-FAs in reducing the damage to organs caused by the particular drug alone. Thus, improvement in the therapeutic response of the drug therapy can improve the clinical outcome.

### DETAILED DESCRIPTION

An exemplary embodiment is directed to a novel injectable drug dosage composition comprising: a sufficient concentration of an oil derived, for example, from fish oil triglycerides containing long-chain omega-3, or n-3, fatty acids (n3-FAs); a drug having an adverse reaction profile that is associated with damage to one or more vital organs; and a water component of an emulsion designed for intravenous injection. An exemplary composition comprises specific and concentrated bioactive n3-FAs for the purpose of addressing particular medical conditions that may be pharmaceutically related (Driscoll, 2009). For example, the bioactive n3-FAs are capable of providing safe treatment of iatrogenic causes of kidney disease, where drug-induced nephrotoxicities are mediated, in part, by reducing blood flow, i.e., ischemia, by altering the vasoconstrictive effects of thromboxane A2. See Driscoll, 2010.

As used herein, the term “oil-containing n3-FAs” pertains to constituents such as triglycerides that are present in marine oils, as well as constituents such as ethyl esters, which are derivatives or products obtained from transesterification of n3-FAs from triglycerides to ester forms. The source of n3-FAs, however, for a given drug formulation can be, for example, wholly natural (for example, unprocessed marine oil) or semi-synthetically derived (for example, processed marine oil). The source of n3-FAs can provide sufficient amounts of bioactive EPA, DHA and/or DPA, as, for example, attached to triglycerides or ethyl ester molecules, in order to mitigate or reduce the adverse effects of a given prescribed drug on a particular vital organ system. The beneficial pharmacological actions attributed to these bioactive n3-FAs include, for example, reductions in oxidative stress, inflammation, immune stimulation and ischemia arising from drug-related injuries.

The provision of bioactive n3-FAs, including EPA, DHA and/or DPA, and the downstream effects on prostaglandin metabolism, as well as the generation of important endogenous chemical mediators from these n3-FA precursors (for example, resolvins and protectins), can potentially have a beneficial effect on the pathophysiological effects of many diseases. This disclosure can extend these potential benefits to apply to selected drugs adversely affecting vital organs of the body. For example, substituting diets that are rich in the omega-6, or n6, fatty acids (linoleic acid and arachidonic acid) with diets rich in n3-FAs (EPA, DHA and/or DPA) can significantly alter the eicosanoid profile (2-series prostaglandins→3-series prostaglandins; 2-series thromboxanes→3 series thromboxanes; 4-series leukotrienes→5-series leukotrienes). For example, prostaglandins of the 2-series derived from n6-FAs are pro-inflammatory compared to 3-series prostaglandins derived from n3-FAs; 2-series thromboxanes obtained from n6-FAs are pro-vasoconstrictive/coagulant compared to 3-series thromboxanes obtained from n3-FAs; and 4-series leukotrienes derived from n6-FAs produce an exaggerated immune response compared to 5-series leukotrienes derived from n3-FAs.

Recent findings regarding chemical mediators (for example, resolvins and protectins) generated from n3-FAs show that the clinical benefits regarding inflammatory processes can extend beyond the initial effect. In addition, these mediators can be actively involved in reducing the extent of oxidative stress as well as facilitating the clearance of debris in the affected areas and reducing the collateral damage to surrounding tissues from an over-exuberant systemic inflammatory response resulting from various physiological causes of tissue injury (for example, infection, trauma, surgery, etc.). Oxidative stress, inflammation, stimulation of the immune response and ischemia can be significant etiological factors involved in pharmacological causes of drug-induced damage to vital organs, and supplying parenteral n3-FAs at the start of medication therapy can substantially reduce these adverse drug effects, as well as reduce or eliminate accompanying morbidity and possible mortality.

In another exemplary embodiment, the n3-FA-containing therapeutic drug vehicle can be used in combination with a prescribed drug intended for intravenous administration. Table 1 provides a broad range of exemplary oil and water ratios in exemplary compositions, along with the corresponding intakes of the bioactive n3-FAs from a 50 mL intravenous drug admixture, as typically used in the clinical setting. For example, the oil to water ratio of the composition can be from about 0.1 to 99.9 to 20.0 to 80.0. The oil to water ratio can depend on, for example, the n3-FA content of the oil phase, the particular at least one drug and marine oil employed, and the particular treatment.

According to another exemplary embodiment, intravenous therapies can provide an increased bioavailability (for example, about 100% of the administered dose) compared to other routes of administration (for example, oral, topical, intramuscular, subcutaneous, suppository, etc.) due to alterations in absorption and/or metabolism of drugs not administered directly into the systemic circulation. As such, the intravenous administration of the therapeutic drug vehicle can increase the rate of incorporation of n3-FAs into plasma membranes (for example, within hours of the infusion) and greatly accelerates the onset of the beneficial effects of n3-FAs compared to the oral, or enteral, route of administration, which can typically include days or weeks of pre-treatment with n3-FAs alone before drug therapy can commence. For example, in a conventional process, when fish oil was used as a vehicle via gastric lavage in an animal model of experimental nephrotoxicity, a 14-day pre-treatment period was necessary to achieve sufficient plasma membrane concentrations to mitigate kidney damage (Elzinga et al, 1987). In a conventional process, in conditions where oral fish oil capsules have been given as therapy, for example, in patients with rheumatoid arthritis and cardiovascular disease, clinical benefits were not apparent until after several months of supplementation. See Calder, 2010b. Thus, bioavailability, and the rapid and successful incorporation of n3-FAs into plasma cell membranes, are crucial in achieving mitigation of adverse drug events when using n3-FA-containing injectable emulsions as a therapeutic drug vehicle.

Another exemplary embodiment is directed to drug candidates prescribed intravenously, for which their use can be associated with significant adverse effects to vital organs, including mechanisms of toxicity involving oxidative stress, inflammation, immune stimulation, and ischemic insult to organ tissues. See Casarett and Doull's Toxicology, The Basic Science of Poisons, 1996. By virtue of administering such drugs intravenously, the high bioavailability engendered therein can increase the toxic potential of these pharmacological agents. Vital organs of the human body can include the brain, heart, lungs, liver and kidneys. For example, the brain is known to be a lipid-rich environment, containing nerve cells and fibers protected by a lipid-containing tissue known as myelin that forms a protective sheath around neuronal structures. A risk of injury is posed by highly lipophilic drugs, such as, for example, the antiarrhythmic agent, amiodarone, a structural analog of the thyroid hormone, which can accumulate in these lipid tissues, destroying the myelin sheath and disrupting nerve conduction. These neurotoxic effects can cause peripheral neuropathy.

Other drugs acting in the central nervous system, such as, for example, levodopa, used in patients with Parkinson's disease, can benefit from n3-FAs. Long-term use of levodopa has been associated with complications in motor function (involuntary movements) that have been linked to high concentrations of arachidonic concentrations in the brain. The provision of n3-FAs can compete with the n6-FA, arachidonic acid, and are the preferred substrate for the important fatty acids in human metabolism. Thus, reducing n6-FA concentrations can be beneficial for patients with Parkinson's Disease in order to reduce the adverse effects of levodopa. See Julien et al., 2006. In another example, the anticancer drug doxorubicin, an anthracycline antibiotic, can cause acute or chronic cardiotoxicity from oxidative stress and the production of reactive oxygen species that induce damage in heart tissues. In laboratory animals, pre-treatment with n3-FA enriched diets for at least 3 weeks has been suggested to improve the therapeutic index of anthracycline antineoplastics. See Germain et al, 2003.

In another example, the antitumor drug, bleomycin, a basic glycopeptide, can induce an oxidative burden on lung tissues, which contain low levels of the drug's inactivating enzyme, bleomycin hydrolase. Increasing lung tissue levels of bleomycin can cause a release of cytokines, for example, tumor necrosis factor, and can also result in interaction with iron and molecular oxygen, which can in turn cause dangerous free radical production. In pulmonary endothelial cell cultures exposed to amiodarone, pre-treatment with n3-FAs was shown to protect against toxicity. See Futamura, 1996. In another example, the anticonvulsant, valproic acid, a branched-chain carboxylic acid, can produce hepatic steatosis, or “fatty liver”, leading to significant liver disease. In an animal model of necroinflammatory liver injury, pre-treatment with n3-FAs reduced oxidative damage and showed protective effects. See González-Periz, 2006. In yet other examples involving animals pre-treated with n3-FAs, several popular antibiotics, such as gentamicin (Priyamvada et al., 2008), and immunosuppressive agents, such as cyclosporine (Yang et al, 2005) were shown to cause less kidney damage. Other drug candidates, that can cause kidney damage, for example, non-steroidal anti-inflammatory agents, including ketorolac and indomethacin, as well as ionic radiocontrast agents, can also benefit from inclusion of n3-FAs in intravenous emulsions containing those drugs or agents.

In exemplary embodiments, co-administration instead of pre-treatment with specific n3-FAs, in high concentration, and in sufficient intravenous doses, using, for example, omega-3 acid-containing marine oils as the therapeutic drug vehicle, can greatly improve the safety profile of parenterally administered drugs that presently exert adverse effects on vital organs. Pre-treatment with n3-FAs before drug therapy is not typically a reasonable option for acutely ill patients requiring drug therapy, for whom fast action can be crucial. Hence, in such cases, supplying n3-FAs through oral or enteral administration is not typically viable or practical. This advantage is especially important in cases where such drug(s) possess a narrow therapeutic index (for example, low ratio of lethal median dose to desirable median dose). The therapeutic index refers to the ratio of the dose required to produce a toxic effect and the dose needed to elicit the desired therapeutic response, and is a relative indication of the potency and safety of the drug. For example, the at least one drug having a narrow therapeutic index can exhibit a significant overlap between the effective dose and the toxic dose.

An example of a drug having a narrow therapeutic index is the aminoglycoside, gentamicin, which is a broad-spectrum parenteral antibiotic against aerobic gram-negative bacteria. An exemplary therapeutic range in plasma is between 4 to 10 μg/mL, but toxicity to the kidneys occurs when the trough blood level (the blood level before the next dose) is above 2 μg/mL. Such exemplary drug has a narrow therapeutic range and the toxicity to kidneys is associated with impaired excretion and drug accumulation. Another example of a drug having a narrow therapeutic index is the antifungal antibiotic amphotericin B which can have a high degree of kidney toxicity, and occurs within the therapeutic dose range. Additional examples of drugs having a narrow therapeutic index include cyclosporine, ketorolac, cisplatin, the anthracycline cancer drug doxorubicin. In the case doxorubicin, a cumulative dose of >550 mg/m² can be associated with cardiomyopathy. In an exemplary embodiment, the use of n3-FAs as a therapeutic drug vehicle with these drugs having a narrow therapeutic index can mitigate the toxic responses to vital organs.

Table 2 depicts examples of possible drugs/categories that can be associated with injury to vital organs. Other drugs/categories can be included where, for example, co-administration of concentrated n3-FAs may accentuate the effects of the primary drug therapy. The examples in Table 2 are not necessarily limiting, but rather are examples of a broad range of possible combinations and permutations.

An exemplary embodiment can employ, for example, a dose range of from about 1 to about 300 mg/kg, as well as combination(s) of n3-FAs designed to accompany a prescribed drug in a proposed intravenous formulation. Table 3 provides examples of the doses (in g of n3-FAs) across the aforementioned dose range for adult patients weighing between 40 and 100 kg. The entries in Table 3 can be applied to lower weights, such as for infants and pediatric patients, where applicable. The examples in Table 3 are not necessarily limiting, but are examples of a broad range of possible combinations and permutations.

According to an exemplary aspect, a source of n3-FAs can be naturally-occurring, semi-synthetic, synthetic, or a combination thereof. For example, a naturally-occurring source of n3-FAs can include fish oil triglycerides. A semi-synthetically-derived source of n3-FAs can include, for example, n3-FAs attached to neutral triglycerides, ethanol as ethyl esters, or a combination thereof. The source of n3-FAs can be naturally-occurring, such as from marine oil triglycerides, but may then be synthetically enriched. The sources of n3-FAs can be from a mixture of naturally-occurring and synthetically-derived products.

For example, an oil that is derived from fish oil can be used which contains n3-FAs at a concentration higher than that occurring in natural sources. The oil can optionally include medium-chain fatty acids from medium chain triglycerides (MCTs), which can be saturated medium-chain fatty acids. The oil can optionally include n6-FAs such as for example, from a vegetable oil. In one embodiment, the composition such as an emulsion can be stable, has normal metabolic clearance, and/or is well-tolerated by patients. For example, the emulsion can be an oil-in-water (o/w) emulsion.

An exemplary oil is derived from fish, and can be rich in the polyunsaturated and bioactive omega-3 fatty acids. The oil component of the emulsion can contain fish oil triglycerides, for example, omega-3 acid triglycerides. The fish oil triglycerides, can be present from about 31% to about 90%, or from about 41% to about 90%, or from about 45% to about 90%, or greater than 50% to about 90%, or from about 51% to about 90%, or from about 55% to about 90%, or from about 60% to about 90%, or from about 70% to about 90%, or from about 80% to about 90%, or from about 40% to about 80%, or from about 50% to about 70%, or from about 60% to about 65%, based on the total weight of the oil component of the emulsion. For example, by employing exemplary ranges of fish oil triglycerides, the amount of esterified omega-3 fatty acids delivered to a human body can be increased. For example, Applicant has recognized the clinical significance of the absolute intake of omega-3 fatty acids, and has discovered that such absolute intake of omega-3 fatty acids can be increased by employing, for example, the exemplary ranges of fish oil triglycerides. For example, Applicants have recognized that in at least some applications, for example cardiovascular health applications, the absolute intake of omega-3 fatty acids can be a more accurate indicator of overall efficacy than the ratio of omega-3 fatty acids to omega-6 fatty acids.

They can be 20- to 22-carbon compounds and can contain 3 or more double bonds located at the 3^{rd} position from the methyl end of the long-chain fatty acid (LCFA) molecule. Standard notation for the various fatty acids (FAs) includes: 1) carbon number, followed by, 2) the number of double bonds, and ending with 3) the position of the double bond relative to the methyl position (or “n3” in the case of the LCFA from fish oil). In particular, the marine oil can be highly enriched with two major n3-FAs, i.e., eicosapentaenoic acid, or EPA (20:5n3), and docosahexaenoic acid, or DHA (22:5n3). The marine oil can contain lesser amounts of other n3-FAs, such as docosapentaenoic acid, or DPA (22:6n3). The fish oil component of the o/w parenteral lipid emulsion can represent oils from a mixture of fatty fish families, such as from the following species: Engraulidae (e.g., anchovies), Carangidae (e.g., mackerel), Clupeidae (e.g., herring), Osmeridae (e.g., smelt), Salmonidae (e.g., salmon) and Scombridge (tuna).

In the European Pharmacopeia (EP), there are two monographs (i.e., EP 1352 entitled “Omega-3 Acid Triglycerides”, and, EP 1912 entitled “Fish Oil, Rich in Omega-3 Acids”) that pertain to fish oil that is acceptable for use in parenteral emulsions (EP 1352, EP 1912, 2008). The monograph EP 1352 substantially differs from EP 1912 in that the composition and requirements for the bioactive n3-FAs in EP 1352 are much higher than in EP 1912 (EP 1352: EPA+DHA≧45%; total n3-FAs≧60% vs. EP 1912: EPA:≧13%; DHA≧9%; total n3-FAs≧28%). The levels of n3-FAs in EP 1912 are consistent with those found in nature. By comparison, in EP 1352, the n3-FA concentrations are substantially higher and can be obtained by an enrichment process such as molecular distillation, whereby certain undesirable fatty acids that are present, for example, myristic acid, palmitic acid and stearic acid, are removed. In so doing, the concentrations of all FAs present, and particularly the n3-FAs, are proportionately elevated (Driscoll, 2008a).

In an exemplary embodiment, the fish oil triglycerides can include omega-3 fatty acids in an amount of at least 60%, based on the total weight of the fatty acids of the fish oil triglycerides. In an exemplary embodiment, the fish oil triglycerides can include a total amount of EPA and DHA of at least 45%, based on the total weight of the fatty acids of the fish oil triglycerides. For example, the fatty acids and omega-3 fatty acids (such as, for example, EPA and DHA) discussed herein refer to the constituent parts of such acids in a fish oil triglyceride, in accordance with EP 1352. For example, the fatty acids and omega-3 fatty acids (such as, for example, EPA and DHA) discussed above can be in their esterified form when present in the fish oil triglycerides.

The fish oil triglycerides can contain at least one n6-FA, for example, a plurality of n6-FAs. The at least one n6-FA can include, for example, arachidonic acid or AA (20:4n6), linoleic acid or LA (18:2n6), alpha linolenic acid or ALA (18:3n3) or a combination thereof. For example, the total content of the at least one n6-FA can be from about 0.1% to about 1.0%, or from about 0.2% to about 0.9%, or from about 0.3% to about 0.8%, or from about 0.4% to about 0.7%, or from about 0.5% to about 0.6%, based on the weight of the oil component of the emulsion.

An exemplary second component of the oil component of the emulsion can include at least one medium chain triglyceride (MCT), for example, a plurality of MCTs. For example, the at least one MCT can be present from about 10% to about 69%, or from about 10% to about 40%, or from about 10% to about 30%, or from about 10% to about 20%, or from about 10% to about 15%, or from about 20% to about 60%, or from about 30% to about 50%, or from about 40% to about 45%, based on the total weight of the oil component of the emulsion. For example, by employing exemplary ranges of MCT, the amount of esterified omega-3 fatty acids delivered to a human body can be increased. For example, by employing exemplary MCT ranges, the amount of esterified omega-3 fatty acids delivered to a human body can be increased with usage of a relatively smaller amount of MCT, while still achieving beneficial metabolic clearance and physicochemical stability characteristics of the emulsion.

For example, the at least one MCT can include a saturated medium chain fatty acid, for example, a plurality of saturated medium chain fatty acids. In an exemplary embodiment, the MCT is a triglyceride of a fatty acid having from 6 to 12 carbon atoms. The MCT can be derived from a plant such as a vegetable, for example, a plurality of plants. The MCT can contain caprylic acid (for example, in an amount of about 50% to about 80% by weight of the MCT), an 8-carbon saturated FA (8:0). The MCT can contain capric acid (for example, in an amount of about 20% to about 50% by weight of the MCT), a 10-carbon saturated FA (10:0). For example, the medium-chain triglycerides can contain triglycerides of caprylic acid and capric acid, in an amount of at least 90% by weight of the medium-chain triglycerides. The description of the MCT for use in this disclosure can, for example, meet the requirements of EP monograph 0868, entitled “Triglycerides, Medium Chain” (Triglycerida saturate media) (EP 0868, 2008).

The content of n3-FAs can be from any single n3-FA, or any combination thereof. In an exemplary embodiment, the composition can contain EPA, DHA, DPA or a combination thereof, for example, each of EPA, DHA and DPA. The individual dosage, for example total daily dosage, of eicosapentaenoic acid (EPA) can vary from 0 to 300 mg/kg of the formulation, for example, from 50 to 250 mg/kg, for example, from 100 to 200 mg/kg, based on the body weight. The individual dosage, for example total daily dosage, of docosahexaenoic acid (DHA) can vary from 0 to 300 mg/kg of the formulation, for example, from 50 to 250 mg/kg, for example, from 100 to 200 mg/kg, based on the body weight. The individual dosage, for example total daily dosage, of docosapentaenoic acid (DPA) can vary from 0 to 300 mg/kg of the formulation, for example, from 50 to 250 mg/kg, for example, from 100 to 200 mg/kg, based on the body weight. For example, EPA, DHA and/or DPA can be present in amounts which are effective to mitigate damage to at least one vital organ which would otherwise be caused by the at least one drug. The individual dosage of n3-FAs can be from any single n3-FA, or any combination thereof (for example containing EPA, DHA and DPA). In an exemplary embodiment, the individual total daily dosage of n3-FAs can be about 1 to about 300 mg/kg, for example, about 100 to 200 mg/kg, based on the body weight.

In another exemplary embodiment, various combinations of the bioactive n3-FAs can be present, with some therapeutic drug vehicles containing specific percentages of selected n3-FAs. In this regard, Table 4 provides a sample of possible n3-FA combinations acting as a therapeutic drug vehicle. For example, EPA can be present in an amount from about 0% to about 100%, for example, from about 30% to about 100%, based on the weight of the total content of n3-FA. For example, DHA can be present in an amount of from about 0% to about 100%, for example, from about 0% to about 30%, based on the weight of the total content of n3-FA. For example, DPA can be present in an amount of from about 0% to about 100%, for example, from about 0% to about 40%, based on the weight of the total content of n3-FA. The examples in Table 4 are not necessarily limiting, but rather are examples of a broad range of possible combinations and permutations.

It is possible in some cases that a specific prescribed drug, within the domain of a defined therapeutic dose, will benefit from or require a specific dose and/or combination of n3-FAs tailored or customized to it in order to maximize the toxicity-mitigating effects of the n3-FAs. Certain prescribed drugs within a category of pharmacological agents can benefit from a particular combination of n3-FAs, or it is possible that such a vehicle can apply to a broad range of drugs and categories, in accordance with an exemplary embodiment. Table 5 provides an example of a therapeutic drug vehicle over several small volume infusions, and the amounts of n3-FAs a patient can receive in a 24 hour period. The examples in Table 5 are not necessarily limiting, but rather are examples of a broad range of possible combinations and permutations.

The concentration of the drug in the composition and the dosage of the drug, for example, total daily dosage, can depend on various factors such as, for example, the n3-FA formulation, the drug and the specific condition being treated. For example, the least one drug can be present in an amount of about 0.005% to about 1.5%, for example, about 0.1% to about 0.5%, based on the weight of the composition. The dosage of the drug can be in an amount of about 0.5 to about 50 mg/kg, for example, about 10 to about 30 mg/kg, based on the weight of the composition. For example, the intravenous volume of a dosage of the composition can be about 25 to about 100 mL/dose for adults, and about 1 to about 10 mL/dose for infants.

As another exemplary embodiment, the prescribed drug can be present in either the oil fraction or the water fraction of an injectable n3-FA-containing oil-in-water emulsion, depending on the physicochemical characteristics of the drug. For example, exemplary compositions and methods can provide for the drug to be entirely present in the oil fraction, entirely present in the water fraction, or present in both the oil and water fractions. For example, this approach can be counter to current practice in the pharmaceutical industry, for example, when using injectable oil-in-water emulsions as a pharmaceutical drug vehicle to safely administer water-insoluble drugs via the intravenous route of administration (Driscoll et al, 2009).

For example, water-insoluble anesthetic/sedative agent propofol, residing in the omega-6 rich oil phase of an injectable oil-in-water emulsion, is an example of conventional practice used in drug vehicle applications by pharmaceutical formulators. In contrast, in an exemplary embodiment, the omega-3 fatty acid-containing oil can function as a novel therapeutic component, as opposed to as merely a pharmaceutical (for example, carrier-only) component. Hence, its use is not limited to a particular group of drugs based on their inherent solubility and partition coefficients with respect to a particular (for example, oil or water) phase of the emulsion. In an exemplary embodiment, the omega-3 fatty acid-containing oil can serve dual purposes, for example, as both a pharmaceutical and therapeutic drug vehicle for selected pharmacological agents.

As another exemplary embodiment, the n3-FAs in a given formulation can accentuate the pharmacological actions of the primary, prescribed drug and improve the therapeutic response to drug therapy. These effects can arise from additive pharmacological effects that both complement the intended actions of the primary drug and also improve and/or accelerate the membrane altering (for example, reparative, sensitization) properties of the n3-FAs. In the first case, for example, the clinical effects of a diuretic such as the “high ceiling, loop diuretic”, furosemide, whose pharmacological actions involve enhanced synthesis of vasodilatory prostaglandins that increase blood flow to the kidneys, can be enhanced by the actions of n3-FAs that form the less vasoconstrictive, thromboxane A3 series. This can be of particular clinical significance in critically ill patients who are fluid-overloaded and resistant to conventional diuretic therapy. In another example, n3-FAs can possess analgesic properties that can complement the actions of drug(s) used in pain management. See Matta et al, 2007. In the second case, for example, it has been suggested that n3-FAs can improve the response to chemotherapy of various cancers by enhanced cytotoxicity of anti-cancer drugs and by reducing oxidative stress in animal and cell culture models (See Abulrob et al, 2000, Rudra et al, 2001, Ding et al, 2004, Calviello et al, 2005, Mahéo et al, 2005, Menendez et al, 2005, Colos et al, 2006, Wang et al, 2006, Manni A et al, 2009,) and humans (See Harries et al, 2005, Bougnoux et al, 2009, Fracasso et al, 2009). In an exemplary embodiment, an additional benefit of using n3-FAs as a therapeutic drug vehicle can be an improvement of clinical outcomes by accentuating the response to primary drug therapy.

In view of the complexity of the diverse actions of n3-FAs that can reduce inflammation, oxidative stress, immune modulation and ischemic injury, and the related pharmacological actions underlying the mechanisms of drug injury to vital organs, there are numerous unique exemplary aspects of this disclosure. Special interactions between n3-FAs and drugs associated with damage to vital organs can result in achieving these benefits from the onset of drug therapy by the intravenous provision of the therapeutic drug vehicle. That is, in an exemplary embodiment, the nearly complete bioavailability of the intravenous route of administration can allow rapid incorporation of n3-FAs into plasma cell membranes to exert mitigation of the toxic effects of selected drugs.

In an exemplary embodiment, having the option to provide n3-FAs in high concentrations far above the levels found in natural marine sources, using semi-synthetic methods of enrichment through attachment to triglyceride or ester molecules, can further enhance their efficient incorporation into plasma cell membranes. For example, a composition described in copending U.S. application Ser. No. 12/382,196 filed on Mar. 11, 2009, and International Application No. PCT/US2010/000723 filed on Mar. 11, 2010, the contents of which are incorporated by reference herein, can be employed in the present compositions and methods. This exemplary advantage can be particularly beneficial, because many intravenous drugs are provided in multiple doses over 24 hours via small-volume parenterals (for example, 100 mL). Hence, in certain cases, using wholly natural sources of fish oil, averaging approximately 30% n3-FAs in the total fatty acid profile, can benefit from or require higher volumes of lipid emulsion per day, which may not be tolerated (for example, inducing hypertriglyceridemia). Also, the use of such relatively low-n3-FA-concentration natural fish oil may be unable to reasonably and safely deliver effective n3-FA doses as a therapeutic drug vehicle at the upper limits indicated in this disclosure (for example, up to 300 mg/kg). Use of exemplary aspects can obviate the concerns associated with pre-treatment with n3-FAs when other routes of administration are applied (e.g., oral or enteral).

In an exemplary aspect, the composition employed in the method can be an emulsion comprising: an oil component and a water component, the oil component comprising: fish oil triglycerides in an amount of about 60% to about 90% based on the weight of the oil component; wherein the fish oil triglycerides comprise omega-3 fatty acids in an amount of at least 60%, based on the total weight of the fatty acids of the fish oil triglycerides; wherein the fish oil triglycerides comprise a total amount of EPA and DHA of at least 45%, based on the total weight of the fatty acids of the fish oil triglycerides; and, at least one medium-chain triglyceride, wherein a total amount of the at least one medium-chain triglyceride is from about 10% to about 40% based on the weight of the oil component.

According to another exemplary aspect, the composition employed in the method can be an emulsion comprising: an oil component and a water component, the oil component comprising: fish oil triglycerides in an amount of greater than 50% to about 90% based on the weight of the oil component of the emulsion; wherein the fish oil triglycerides comprise omega-3 fatty acids in an amount of at least 60%, based on the total weight of the fatty acids of the fish oil triglycerides; wherein the fish oil triglycerides comprise a total amount of EPA and DHA of at least 45%, based on the total weight of the fatty acids of the fish oil triglycerides; and, a medium-chain triglyceride.

According to another exemplary aspect, the composition employed in the method can be an emulsion comprising: an oil component and a water component, the oil component comprising: fish oil triglycerides in an amount of about 31% to about 90% based on the weight of the oil component of the emulsion; wherein the fish oil triglycerides comprise omega-3 fatty acids in an amount of at least 60%, based on the total weight of the fatty acids of the fish oil triglycerides; wherein the fish oil triglycerides comprise a total amount of EPA and DHA of at least 45%, based on the total weight of the fatty acids of the fish oil triglycerides; and, a medium-chain triglyceride; wherein the emulsion is an oil-in-water emulsion, and wherein the concentration of the oil component in the emulsion is 5 g/100 mL to less than 20 g/100 mL, or the concentration of the oil component in the emulsion is greater than 20 g/100 mL to 30 g/100 mL.

In an exemplary embodiment, n3-FAs can, for example, exert their beneficial effects by modifying the common mechanisms of tissue injury underlying drug toxicity to vital organs. Fourth, by reducing the toxic potential of drugs on vital organs, higher doses of certain drug(s) can be given in order to address the underlying clinical problem, which can increase the clinical efficacy of certain drug regimens in a dose-dependent manner. Fifth, in selected cases, n3-FAs can improve the therapeutic response of drugs by accentuating or complementing their mechanisms of pharmacological actions.

The therapeutic drug vehicle can exert its toxicity-mitigating effects of selected drugs by reducing oxidative stress, reducing inflammation, adverse immune responses, reducing ischemia, or a combination thereof. The composition of the n3-FA-containing therapeutic vehicle can be tailored to a specific drug, a specific dose of a drug, several drugs in the same therapeutic category, and/or several drugs spanning several therapeutic categories. The therapeutic drug vehicle can accentuate the beneficial pharmacological effects of the drug in the formulation in addition to mitigation of its toxicity. The therapeutic drug vehicle can accentuate the beneficial pharmacological effects of the drug in the formulation that can reduce the amount of drug necessary with a further mitigation in its toxicity. The therapeutic drug vehicle can improve the therapeutic response of drug therapy and thus, clinical outcome by way of its reparative properties. The therapeutic drug vehicle can be used for parenteral administration of drugs as, for example, an oil-in-water injectable emulsion so as to exert its beneficial effects at the onset of drug therapy. The addition of the drug to a therapeutic drug vehicle produced from this application can reside in either the “dispersed” or “internal” phase or in the “continuous” or “external” phase of an emulsion formulation.

In an exemplary embodiment of a method of parenterally administering the composition, the at least one omega-3 fatty acid and the at least one drug are administered simultaneously. For example, such simultaneous administration can be achieved by virtue of the at least one omega-3 fatty acid and the at least one drug being present in the same emulsion composition. Any suitable parenteral administration can be used including, for example, intravenous administration and/or intra-arterial administration.

In an exemplary embodiment, the method does not include a pretreatment process of pretreating the person with an omega-3 fatty acid prior to the step of parenterally administering the composition. For example, the pretreatment process that is excluded according to an exemplary embodiment can include the daily administration of an omega-3 fatty acid. For example, the pretreatment process that is excluded according to an exemplary embodiment is a pretreatment with an omega-3 fatty acid that occurs 1 day or more prior to administration of the composition or, for example, 3 days or more prior to administration or, for example, 7 days or more prior to administration or, for example, 14 days or more prior to administration. For example, the pretreatment process that is excluded according to an exemplary embodiment is a pretreatment with an omega-3 fatty acid that occurs 3 to 21 days prior to administration of the composition or, for example, 7 to 14 days prior to administration of the composition.

TABLE 1
Examples of various Emulsion Mixtures (Oil:Water or O:W
Ratios) and Corresponding n3-FA Intakes from a 50 mL Small-
Volume Drug Admixture Dose

| | n3-FA Content of the Oil Phase | | | | |
|---|---|---|---|---|---|
| O:W Ratio | 20% | 40% | 60% | 80% | 100% |
| (g of oil/dose) | (g of n3-FA/dose) | | | | |
| 0.1:99.9 (0.05) | 0.01 | 0.02 | 0.03 | 0.04 | 0.05 |
| 0.5:99.5 (0.25) | 0.05 | 0.10 | 0.15 | 0.20 | 0.25 |
| 1.0:99.0 (0.50) | 0.10 | 0.20 | 0.30 | 0.40 | 0.50 |
| 5.0:95.0 (2.50) | 0.50 | 1.00 | 1.50 | 2.00 | 2.50 |
| 10.0:90.0 (5.0 g) | 1.00 | 2.00 | 3.00 | 4.00 | 5.00 |
| 15.0:85.0 (7.5 g) | 1.50 | 3.00 | 4.50 | 6.00 | 7.50 |
| 20.0:80.0 (10 g) | 2.00 | 4.00 | 6.00 | 8.00 | 10.00 |

TABLE 2
Examples of Potential Drugs/Categories Affecting Vital Organs
That May Benefit From n3-FA Damage Mitigation Therapy

| | |
|---|---|
| 1. | Antibiotics |

| | |
|---|---|
| a. | aminoglycosides |
| b. | amphotericin |
| c. | chloramphenicol |
| d. | ketoconazole |
| e. | macrolides |
| f. | quinolones |
| g. | tetracyclines |

| | |
|---|---|
| 2. | Antineoplastic Agents |

| | |
|---|---|
| a. | alkylating agents |
| b. | antimetabolites |
| c. | antimitotics platinum coordination complexes |

| | |
|---|---|
| 3. | Anti-Parkinson Agents |

| | |
|---|---|
| a. | levodopa |
| b. | pramipexole |
| c. | ropinirole |
| d. | rotigotine |
| e. | bromocriptine |

| | |
|---|---|
| 4. | Cardiovascular Agents |

| | |
|---|---|
| a. | adenosine |
| b. | amiodarone |
| c. | angiotensin converting enzyme (ACE) inhibitors |
| d. | flecainide |

| | |
|---|---|
| 5. | Diuretics |

| | |
|---|---|
| a. | loop diuretics |
| b. | potassium-sparing diuretics |
| c. | thiazides |

| | |
|---|---|
| 6. | Immunosuppressive Agents |

| | |
|---|---|
| a. | Azathioprine |
| b. | Cyclosporine |
| c. | Mycophenolate |
| d. | Tacrolimus |

| | |
|---|---|
| 7. | Non-Steroidal Anti-Inflammatory Drugs (NSAIDs) |

| | |
|---|---|
| a. | acetaminophen |
| b. | aspirin |
| c. | ibuprofen |
| d. | indomethacin |
| e. | ketorolac |

| | |
|---|---|
| 8. | Psychotropics |

| | |
|---|---|
| a. | haloperidol |
| b. | monoamine oxidase inhibitors |
| c. | phenothiazines |
| d. | serotonin reuptake inhibitors |
| e. | thioxanthines |

TABLE 3
Intakes of n3-FAs (g/dose) Ranging from 1 to 100 mg/kg
n3-FA Dose Range, mg/kg

| | | | |
|---|---|---|---|
| 1 | 10 | 50 | 100 |

| | |
|---|---|
| Adult Patient | g n3-FA/ |
| Weight, kg | body weight |

| | | | | |
|---|---|---|---|---|
| 40 | 0.04 | 0.4 | 2.0 | 4.0 |
| 50 | 0.05 | 0.5 | 2.5 | 5.0 |
| 60 | 0.06 | 0.6 | 3.0 | 6.0 |
| 70 | 0.07 | 0.7 | 3.5 | 7.0 |
| 80 | 0.08 | 0.8 | 4.0 | 8.0 |
| 90 | 0.09 | 0.9 | 4.5 | 9.0 |
| 100 | 0.10 | 1.0 | 5.0 | 10.0 |

TABLE 4
Sample of Potential n3-FAs and Dose Ranges (% of n3-FA Oil
Profile) as Therapeutic Drug Vehicles

| EPA | DHA | DPA |
|---|---|---|
| 100 | 0 | 0 |
| 80 | 20 | 0 |
| 60 | 40 | 0 |
| 40 | 60 | 0 |
| 20 | 80 | 0 |
| 0 | 100 | 0 |
| 0 | 80 | 20 |
| 0 | 60 | 40 |
| 0 | 40 | 60 |
| 0 | 20 | 80 |
| 0 | 0 | 100 |
| 20 | 0 | 80 |
| 40 | 0 | 60 |
| 60 | 0 | 40 |
| 80 | 0 | 20 |
| 10 | 80 | 10 |
| 20 | 60 | 20 |
| 30 | 40 | 30 |
| 40 | 30 | 30 |
| 60 | 20 | 20 |
| 10 | 10 | 80 |
| 20 | 20 | 60 |
| 20 | 40 | 40 |
| 30 | 30 | 40 |

TABLE 5
Small-Volume Parenteral Infusions and n3-FA Intakes Using a
10% Oil-in-Water Emulsion with 50% n3-FAs in the Oil Phase
Infusions per Day

| | | | |
|---|---|---|---|
| 1 | 2 | 3 | 4 |

| | |
|---|---|
| Infusion Volume | (g n3-FA) |

| | | | | |
|---|---|---|---|---|
| 1 mL | 0.05 | 0.10 | 0.15 | 0.20 |
| 5 mL | 0.25 | 0.50 | 0.75 | 1.00 |
| 10 mL | 0.50 | 1.00 | 1.50 | 2.00 |
| 25 mL | 1.25 | 2.50 | 3.75 | 5.00 |
| 50 mL | 2.50 | 5.00 | 7.50 | 10.00 |

While various embodiments are described herein, it will be appreciated that variations, modifications and other changes in form and detail may be made without departing from the spirit and scope of the disclosure. Such variations and modifications are to be considered within the purview and scope of the disclosure as defined by the appended claims.

1. A method of parenterally administering a composition, the method comprising parenterally administering to a person a composition comprising at least one omega-3 fatty acid and at least one drug, wherein the at least one omega-3 fatty acid and the at least one drug are administered simultaneously.

2. The method according to claim 1, wherein the at least one omega-3 fatty acid is obtained from a marine oil, and wherein the at least one omega-3 fatty acid is in a naturally occurring form.

3. The method according to claim 1, wherein the at least one omega-3 fatty acid is obtained from a marine oil, and wherein the at least one omega-3 fatty acid is in a non-naturally occurring form.

4. The method according to claim 1, wherein the at least one omega-3 fatty acid is obtained from a marine oil, and wherein the at least one omega-3 fatty acid is attached to neutral triglycerides, ethanol as ethyl esters or a combination thereof.

5. The method according to claim 1, wherein the at least one omega-3 fatty acid comprises eicosapentaenoic acid, docosahexaenoic acid and docosapentaenoic acid.

6. The method according to claim 5, wherein the eicosapentaenoic acid is present in an amount of 30% or greater, the docosahexaenoic acid is present in an amount of 30% or less, and the docosapentaenoic acid is present in an amount of about 40% or less, based on the weight of the total omega-3 fatty acid content.

7. The method according to claim 5, wherein a total daily dosage of the at least one omega-3 fatty acid is about 1 to about 300 mg/kg, based on the weight of the total omega-3 fatty acid content.

8. The method according to claim 1, wherein the composition is in the form of an emulsion comprising an oil phase and a water phase, wherein the at least one omega-3 fatty acid and the at least one drug are present in the oil phase.

9. The method according to claim 1, wherein the method does not include a pretreatment process of pretreating the person with an omega-3 fatty acid source prior to parenterally administering the composition.

10. The method according to claim 1, wherein the at least one drug is a material that damages a vital organ when the material is not simultaneously administered with the at least one omega-3 fatty acid.

11. The method according to claim 1, wherein the at least one drug is selected from the group consisting of an amphotericin, quinolone, antineoplastic agent, amiodarone, loop diuretic, azathioprine, cyclosporine, tacrolimus, indomethacin, ketorolac and a combination thereof.

12. The method according to claim 1, wherein the at least one omega-3 fatty acid is present in an amount that is effective to reduce or eliminate at least one adverse drug effect selected from the group consisting of oxidative stress, inflammation, immune stimulation, ischemia of at least one vital organ, and a combination thereof.

13. The method according to claim 1, wherein the at least one drug is present in an amount of about 0.005% to about 1.5%, based on the weight of the composition.

14. The method according to claim 1, wherein a dosage of the drug is in an amount of about 0.5 to about 50 mg/kg, based on the weight of the composition.

15. The method according to claim 1, wherein the parenteral administration comprises intravenous administration.
